(19) United States
(12) Patent Application Publication
(10) Pub. No.: US 2023/0293280 A1
(43) Pub. Date: Sep. 21, 2023

(54) Helical Tissue Cutter and Trocars Attachable to Common Handle for Mesh Sling Deployment and Myomectomy

(71) Applicant: Jamil Mogul, Santa Clara, CA (US)

(72) Inventor: Jamil Mogul, Santa Clara, CA (US)

(21) Appl. No.: 18/165,833

(22) Filed: Feb. 7, 2023

Related U.S. Application Data
(60) Provisional application No. 63/321,746, filed on Mar. 20, 2022.

(51) Int. Cl. A61F 2/00; A61B 17/34

(52) U.S. Cl. CPC A61F 2/0063; A61B 17/3468; A61F 2002/0072; A61B 2017/320056

(57) ABSTRACT

A helical tissue cutter, used for safely and speedily resecting biological tissues and spiraling out the resected tissues and any biological debris, having an enclosed, exposable, retractable cutter with a helical blade that is continued further as a helical fin; a tubular curved trocar having a capability and means to intravaginally carry, deploy and mechanistically release a mesh sling attached to an anchor using a retropubic approach to treat stress urinary incontinence; a turnable trocar that can be used on the right or left side of the groin to deploy a mesh sling by applying a transobturator approach to treat women's stress urinary incontinence; and a handle having a motorized capability and means to couple, attach, handle, operate, and retract the helical tissue cutter while having an aspiration connection for a vacuumized tissue collector, or to couple, attach, handle, and operate the tubular curved trocar or the turnable trocar.

### CROSS REFERENCE TO RELATED APPLICATIONS

Applicant claims priority of provisional application 63/321,746, filed Mar. 20, 2022, for “Tissue Cutter for Myomectomy” and provisional application 63/321,744, filed Mar. 20, 2022, for “Sling Mesh Introducer for Stress Urinary Incontinence”,

### BACKGROUND

### Field of the Invention

The present invention relates to an apparatus that resects intravaginal tissues to create periurethral tunnels for deployment of a mesh sling, that also resects uterine fibroid tissues via the vaginal orifice for myomectomy, and that deploys the mesh sling intravaginally via a retropubic or transobturator approach to treat stress urinary incontinence.

### Prior Art

Stress urinary incontinence in women occurs when the supports of the urethra are weakened allowing for urethral hypermobility or instability, which causes involuntary leakage of urine related to an increase in intraabdominal pressure that occurs during sneezing, laughing, coughing or exercise. To treat stress urinary incontinence, a mesh sling to support the midurethra and ultimately decrease hypermobility or instability is placed using a retropubic approach, in which the mesh sling curves around the pubic bone and exits through the suprapubic area. The retropubic midurethral mesh sling procedure is performed intravaginally by creating periurethral tunnels via a sharp surgical tool and accessories and by making two suprapubic incisions, and then by using a curved trocar to carry and place the mesh sling under the midurethra in the tunnels and to exit the suprapubic incisions where the ends of the mesh sling are trimmed off at the skin's surface. A cystoscopy is performed afterwards to confirm that no injury to the bladder, bowel, blood vessels or nerves occurred or sustained.

Alternatively, a small incision is made in the vagina under the urethra, and one small incision is made on the left side or fold of the groin and another small incision is made on the right side or fold of the groin and through the obturator foramen, the space created by the ischium and pubis pelvic bones. A right curved trocar is inserted into the incision on the right side of the groin and advanced through the obturator canal of the pelvis and into the incision in the vagina. This step is repeated on the left side of the groin using a left curved trocar. A mesh sling is then attached to the right and left curved trocars on each end, which are then withdrawn pulling the mesh sling into place under the urethra. The trocars are removed, the mesh tension is adjusted, and the excess mesh sling is trimmed off at the skin's surface. It is called a transobturator approach.

Myomectomy is a procedure to remove fibroids (also known as leiomyomas) from the uterus, which are non-cancerous growths made up of connective tissues and muscle cells and which can cause pelvic pain, irregular bleeding or bleeding between periods, heavy menstrual bleeding, and/or an inability to fully empty the bladder. Fibroids are removed by excising the fibroid fully at once or layer by layer in steps using a moving and mechanized tissue cutter in the myomectomy procedure performed openly or abdominally, minimally invasively (laparoscopically or robotically), or hysteroscopically (via the vaginal orifice).

The Problems Addressed

During the retropubic mesh sling procedure of treating stress urinary incontinence, bare sharp surgical tools with often sharp accessories used for creating periurethral tunnels can cause injuries or perforations to the bladder, bowel, blood vessels or nerves, which requires cystoscopy after the deployment of the mesh sling to confirm that no injury occurred. Also, these manual surgical tools and accessories are for resecting tissues little by little, which is tedious and time-consuming, and their inadvertent use can lead to other complications, such as excessive bleeding or urinary tract or wound infections.

For the transobturator mesh sling procedure, which has fewer complications, two trocars, each with a curve but one in the opposite direction, are used. One trocar is used for the left side of the groin while other trocar is used for the right side of the groin, making the surgeon be mindful of or remember which is which, adding an extra alertness during the procedure, complicating the sorting and tracking of the pair of the left and right curved trocars, and causing a purchase of the trocars in pairs if one of the two trocars breaks as they are sold in left-and-right pairs, which is a waste of money.

Per various studies, the rates of all complications to the patients including also injuries, infections and perforations are up to 15.6% for the retropubic mesh sling procedure and 8.9% to 9.2% for the transobturator mesh sling procedure.

For a hysteroscopic myomectomy procedure, an electrically-motorized tubular tissue cutter, which is connected via an aspiration tubing attached to a vacuumized tissue collector, is inserted into the working channel of a hysteroscope that is inserted into the vaginal orifice, and the distal end of the tissue cutter is advanced into the uterine and made in contact with the uterine fibroids that are removed by excising each fibroid a layer by layer in steps. Sometimes the resected or partially resected tissues of the fibroids get stuck at or inside the tubular tissue cutter resulting in jamming or ceasing of the cutter. This also adds to dullness of the cutter and poor aspiration of the resected tissues and/or biological debris, which often causes clogging of the aspiration tubing, resulting in a drastically increased time to remove all the fibroids. Either jamming or clogging, in turn, possibly increases an injury risk to nearby healthy tissues or blood vessels and increases the cost due to the increased procedure time.

A Solution to the Problems

To address the issues mentioned above, a tissue cutter is designed, which is attachable and detachable and which is protective, mechanized and motorized to safely and speedily resect the intended tissue without a collateral damage to the surrounding tissues or nerves, or any nearby vessels in order to create periurethral tunnels for placing the mesh sling; and a curved trocar is designed, which is attachable and detachable and which can be axially rotated to a 180-degree turn and locked in place to function as a right or left curved trocar or unlocked and turned in the opposite direction and relocked in place. Additionally, for the retropubic approach, a tubular curved trocar is designed, which quickly and mechanistically releases the attached mesh sling after its deployment. The protective tissue cutter is also designed to resect all types and sizes of uterine fibroids safely and swiftly for the hysteroscopic myomectomy procedure.

### BRIEF SUMMARY OF THE INVENTION

In accordance with the present invention, a brief summary of the invention is stated below.

A helical tissue cutter with an elongated axial shaft, which is rotary, slidable and retractable, and with a sharp-edged helical blade that continues further as a flat-edged helical fin around the shaft, is enclosed longitudinally in a protective hollow cylinder that is attachable to and detachable from the front end of a handle that contains a boxed spring-wound motor, gears and a retractable sliding mechanism, to which the cutter's shaft is coupled and decoupled, respectively, during the attachment and detachment of the cutter. The hollow cylinder, which also acts as an aspiration channel, has at least two elongated through slots, one on each side around the hollow cylinder's distal end where the cutter resides and is intentionally exposed when the cutter is slid all the way toward the slotted distal end of the hollow cylinder by a spring-loaded retractable slider knob on the handle. There is also a motor crank knob on the back end of the handle, which knob is turned to power the boxed spring-wound motor in order to rotationally drive the coupled cutter that not only resects the tissues but also, thanks to the cutter's helical shape, spirals out the resected tissues. To aspirate the resected and spiraled-out tissues and/or any biological debris, a standard vacuumized tissue collector containing a standard aspiration tubing is connected to the back end of the handle, which end has a standard luer connector for the aspiration tubing.

To create periurethral tunnels for intravaginally placing a midurethral mesh sling or for resecting tissues to remove uterine fibroids or to perform myomectomy while the patient is under anesthesia, the helical tissue cutter is introduced into the vagina and deployed at the tissue site. The tissue resecting process starts by turning the motor crank knob fully and then by sliding the slider knob all the way to the front and holding the slider knob. After the tissue resecting process is completed, the hold on the slider knob is lifted and the cutter automatically slides back out of the slotted end. Then the helical tissue cutter is detached, and the aspiration tubing is disconnected from the handle.

Using the retropubic approach to intravaginally place a midurethral mesh sling to treat stress urinary incontinence, a tubular curved trocar, which has a receptacle feature at its distal end for an anchor that is adhered to either end of a mesh sling, is attached from the trocar's proximal end to the front end of the handle; the anchor with the mesh sling is inserted into the receptacle feature at the distal end of the curved trocar; and while the patient is under anesthesia, the mesh sling placement procedure is started. To quickly release the mesh sling from the trocar after mesh sling deployment, the spring-loaded retractable slider knob on the handle is moved forward, dislodging the anchor with the mesh sling from the trocar.

Using the transobturator approach to place a urethral mesh sling to treat stress urinary incontinence, a turnable trocar, which is curved, is attached to the front end of the handle and one end of the mesh sling is engaged at the distal tip of the turnable trocar. While the patient is under anesthesia, the incisions are made, and then the turnable trocar along with the engaged mesh sling is inserted into the vagina to perform the transobturator sling procedure, in which the same trocar is used for either the left side or the right side by turning the trocar to a 180° angle that is locked in place after completing the procedural steps on one side.

An important advantage of the present invention is that it has a protective, mechanized and motorized helical tissue cutter that safely and speedily removes uterine fibroids or creates periurethral tunnels for intravaginally placing a midurethral mesh sling using the retropubic approach to treat stress urinary incontinence, eliminating the tedious and time-consuming aspects of creating periurethral tunnels and reducing the risks of traumas, injuries or perforations to the bladder, bowel, blood vessels or nerves.

Another important advantage of the present invention is that it simultaneously spirals out the resected tissues and/or biological debris due to the helical shape of the cutter, which doubly resects the tissues in one turn through the two opposing side slots of the hollow cylinder, avoiding any jamming or ceasing of the cutter and further aiding in optimal aspiration of the resected tissues and/or biological debris. It drastically decreases the time to resect all the targeted tissues to create periurethral tunnels or to remove fibroids, decreases an injury risk to nearby healthy tissues or blood vessels, and reduces the costs of the retropubic mesh sling and hysteroscopic myomectomy procedures due to the decreased procedure time.

Yet another important the present invention is that it quickly and mechanistically releases the anchored mesh sling after its deployment intravaginally using the retropubic approach to treat stress urinary incontinence, making the procedure simpler, easier and faster, and saving time and money as a result.

Still another important advantage of the present invention is that it has only one curved trocar, which can be used for either right or left side of the groin by turning the curve to a 180° angle and locking the curve for placing a urethral mesh sling using the transobturator approach to treat stress urinary incontinence, creating an ease in the procedure, simplifying the inventory and tracking of the trocars, and saving money.

These and other advantages of the present invention will become apparent to those skilled in the art after a reading of the following detailed disclosure of the embodiment of the present invention.

### BRIEF DESCRIPTION OF THE VIEWS IN THE DRAWING

FIG. 1A, FIG. 1B, FIG. 1C, and FIG. 1D respectively show left-side, top, right-side, and cut-away section views of the helical tissue cutter with the receptacle luer lock and the cutter's coupling feature while FIG. 1E, FIG. 1F, FIG. 1G, FIG. 1H, and FIG. 1J show enlarged broken views of the details of the helical tissue cutter with the receptacle luer lock and the cutter's coupling feature.

FIG. 2A, FIG. 2B, and FIG. 2C respectively show left-side, top and front views of the assembled handle while FIG. 2D and FIG. 2G respectively show a cut-away section of the left-side view and a top view of the opened handle and FIG. 2H, FIG. 2J and FIG. 2K show their enlarged broken views of the details containing the boxed spring-wound motor, the gears, coupler shaft, sliding mechanism, extension spring, the shaft's coupling feature, the plug luer lock, the aspiration channel with the receptacle luer lock, and the slider and crank knobs; and FIG. 2E and FIG. 2F show enlarged broken views of the details of the shaft's coupling feature.

FIG. 3A shows a top view of the assembled handle with the helical tissue cutter attached to the handle via the plug luer lock on the Y-branched aspiration channel and coupled to the coupler shaft while FIG. 3B shows an enlarged broken view of the details of the coupling; FIG. 3C shows a top view of the assembled handle with the helical tissue cutter attached and coupled and in the retracted state; FIG. 3D shows a top view of the opened handle with the helical tissue cutter attached and coupled and in the retracted state; and FIG. 3E and FIG. 3F respectively show enlarged broken top views of the details of the retracted helical tissue cutter and the opened handle.

FIG. 4A, FIG. 4B, and FIG. 4C respectively show left-side, top, and right-side views of the hollow cylinder.

FIG. 5A and FIG. 5B respectively show a top view of the helical tissue cutter and an enlarged broken view of the details of the helical tissue cutter.

FIG. 6A and FIG. 6B respectively show top and front views of the coupler shaft for the helical tissue cutters while FIG. 6C and FIG. 6D respectively show enlarged broken views of the details of the coupling feature.

FIG. 7A, FIG. 7B, and FIG. 7C respectively show left-side, front, and right-side views of the cutter gear.

FIG. 8A and FIG. 8B respectively show left-side and front views of the elongated motor drive gear.

FIG. 9A and FIG. 9B respectively show left-side and top views of the boxed spring-wound motor only showing the motor's extended drive shaft and crank shaft.

FIG. 10A and FIG. 10B respectively show top and front views of the elastomeric seal plug.

FIG. 11A and FIG. 11B respectively show front and bottom views of the crank knob.

FIG. 12A, FIG. 12B, FIG. 12C, and FIG. 12D respectively show front, left-side, bottom, and right-side views of the slider knob.

FIG. 13 shows a top view of the Y-branched aspiration channel.

FIG. 14A, FIG. 14B, and FIG. 14C respectively show top, right-side and backside views of the bottom part of the handle.

FIG. 15A and FIG. 15B respectively show top and right-side views of the top part of the handle.

FIG. 16 shows a front view of the mesh sling with the anchors glued to either end of the mesh sling.

FIG. 17A and FIG. 17B respectively show front and right-side views of the anchor while FIG. 17C shows an enlarged broken view of the details of the anchor.

FIG. 18 shows a top view of the assembled handle with the slider knob that is slid to a certain distance.

FIG. 19 shows a top view of the tubular curved trocar.

FIG. 20A shows a top view of the tubular curved trocar that is attached to the assembled handle and coupled to the sliding mechanism via the coupler shaft contained inside the handle, while FIG. 20B shows an enlarged broken view with a cut-away section of the details of the installed anchor containing the mesh sling.

FIG. 21A shows a top view of the curved tube while FIG. 21B shows an enlarged broken view of the details of the distal end of the curved tube.

FIG. 22 shows a top view of the slidable shaft in a straight position, which shaft goes into the curved tube and conforms to the curve of the curved tube.

FIG. 23A and FIG. 23B respectively show front and top views of the turnable trocar.

FIG. 24 shows a top view of the turnable trocar attached to the assembled handle.

FIG. 25A shows a top view of the turnable trocar's shaft while FIG. 25B shows an enlarged broken view with a cut-away section of the details of the turn-lock mechanism installed at the distal end of the turnable trocar's shaft.

FIG. 26A shows a front view of the curved trocar wire having the stem while FIG. 26B shows an enlarged broken view with a cut-away section and FIG. 26C and FIG. 26D show enlarged broken views of the details of the stem of the curved trocar wire.

FIG. 27 shows a top view of the shaft of the turnable trocar.

FIG. 28A, FIG. 28B, and FIG. 28C respectively show left-side, front and right-side views of the receptacle stud that houses the turn-lock mechanism.

### DETAILED DESCRIPTION OF THE INVENTION

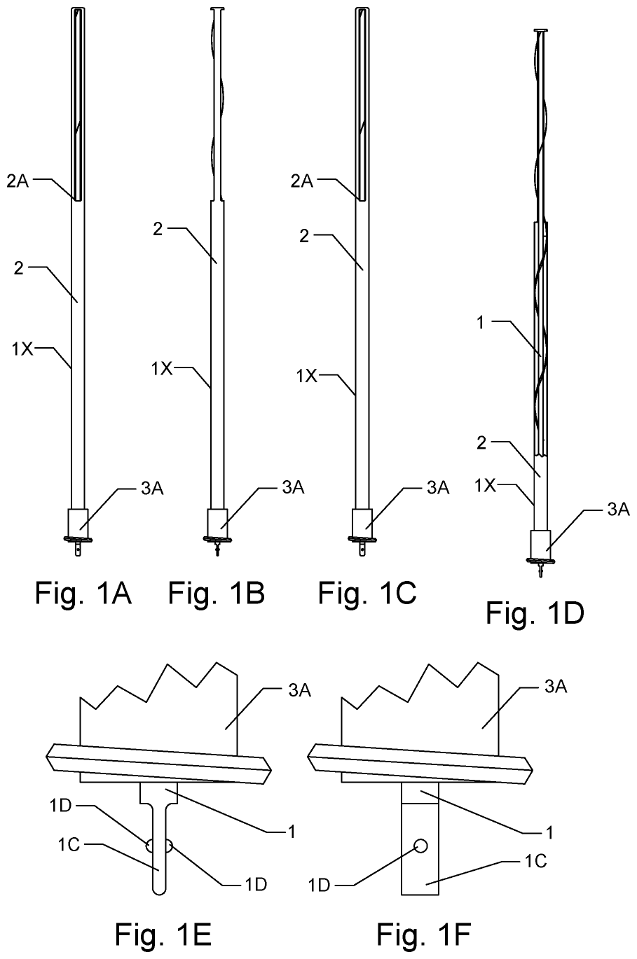

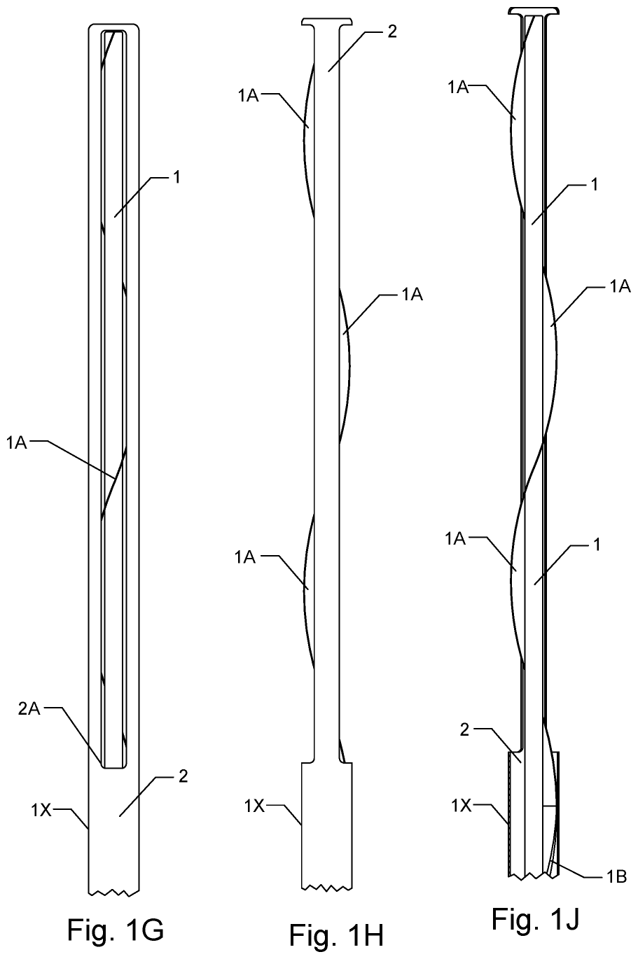

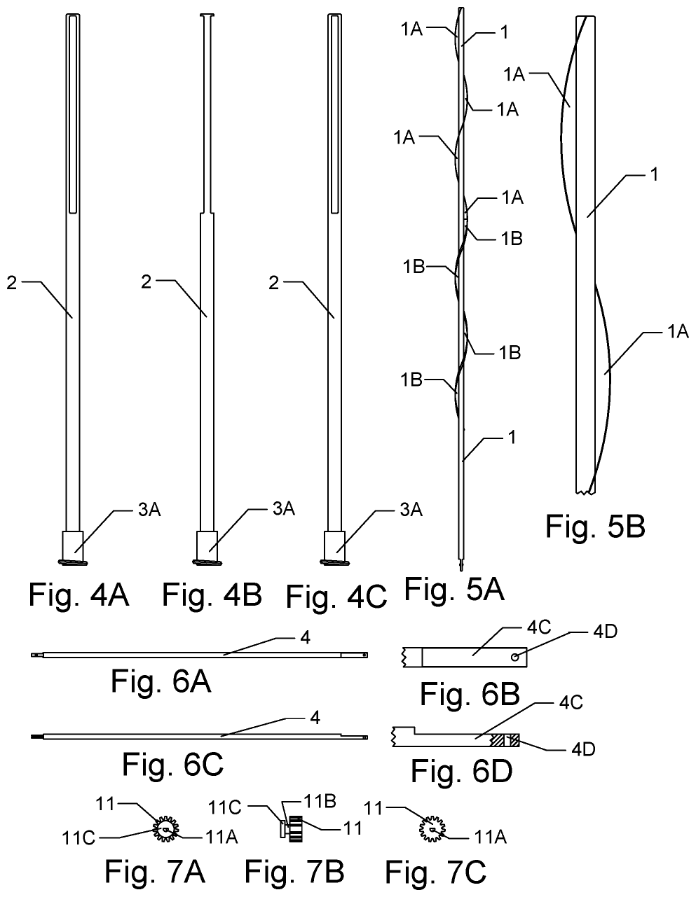

Now, referring to FIG. 1A, FIG. 1B, FIG. 1C, FIG. 1D, FIG. 1E, FIG. 1F, FIG. 1G, FIG. 1H, FIG. 1J, FIG. 4A, FIG. 4B, FIG. 4C, FIG. 5A, FIG. 5B, FIG. 6A, FIG. 6B, FIG. 6C, and FIG. 6D in accordance with the present invention, a helical tissue cutter 1X consists of a cutter shaft 1, and a hollow cylinder 2. For resecting biological tissues and spiraling out the resected tissues and any biological debris, the cutter shaft 1 distally has a sharp-edged helical blade 1A, which blade is longitudinally spiraled around and integral to the cutter shaft 1 and has continuously another matching flat-edged helical fin 1B, which fin is also longitudinally spiraled around and integral to the cutter shaft 1. The cutter shaft 1 proximally has a flattened end 1C and on either side of the flattened end 1C has a rounded boss 1D, which boss performs a coupling function by engaging. The hollow cylinder 2 encloses the cutter shaft 1 containing the sharp-edged helical blade 1A and the flat-edged helical fin 1B. The hollow cylinder 2 is also used for aspiration of the resected tissues and/or biological debris. To expose the sharp-edged helical blade 1A, the hollow cylinder 2 has at least two elongated slots 2A longitudinally, one on either aspect of the hollow cylinder 2, which slots are located 180° apart. For an attachment purpose, the hollow cylinder 2 integrally contains on one end a receptacle luer lock 3A. The other end of the hollow cylinder 2 is open and has a bent and rounded edge.

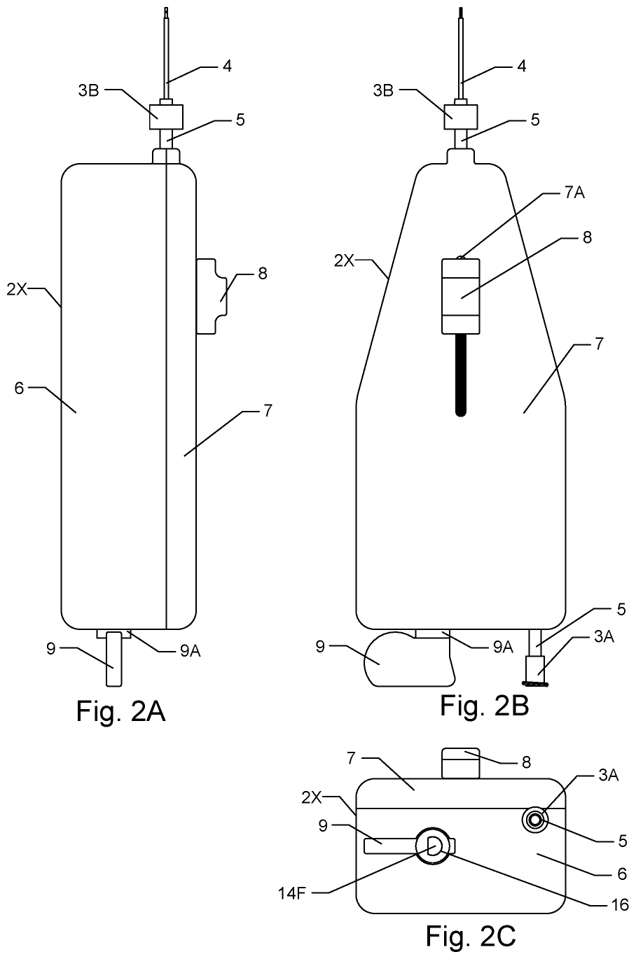

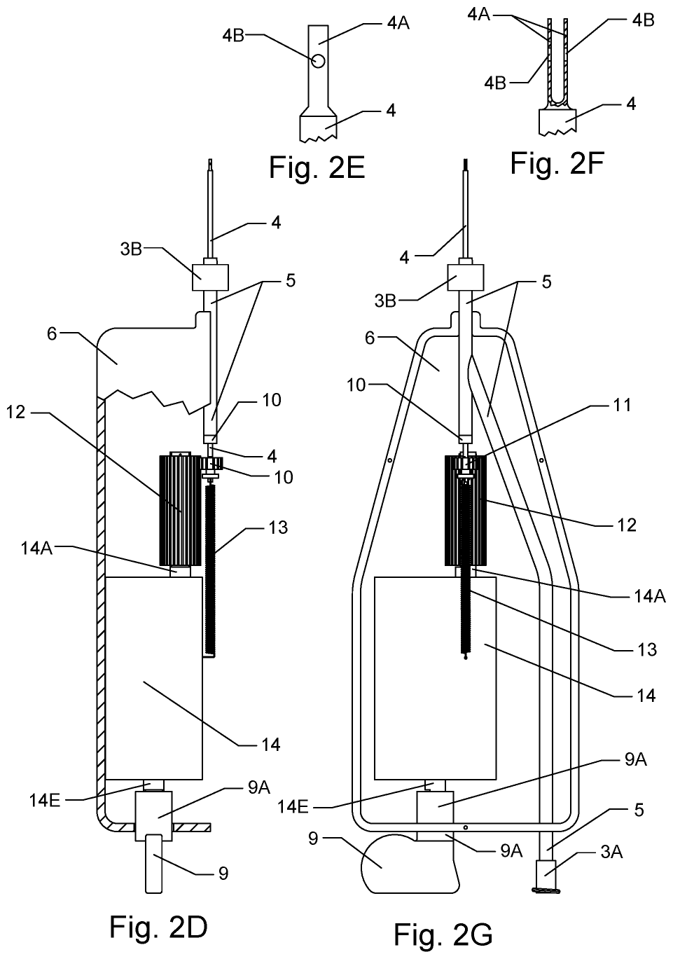

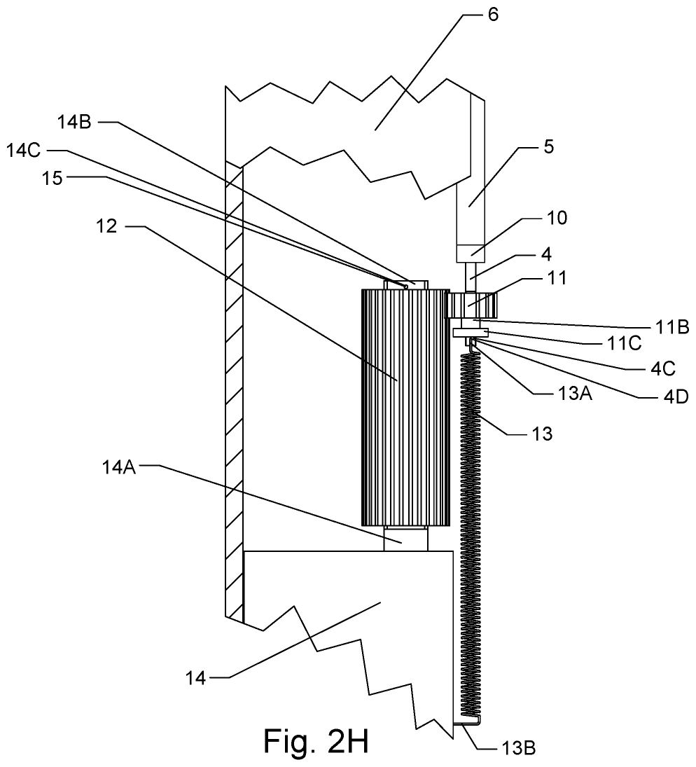

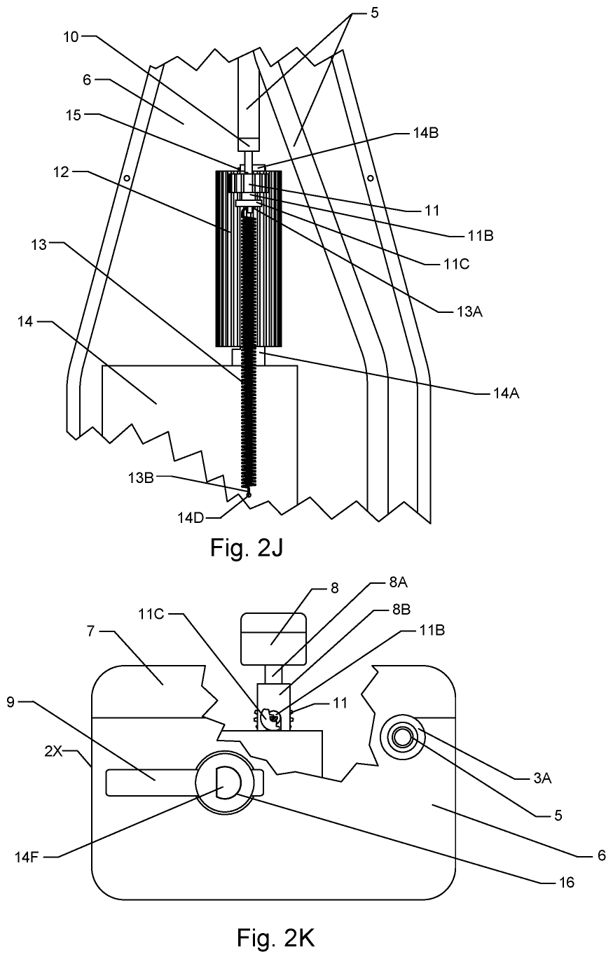

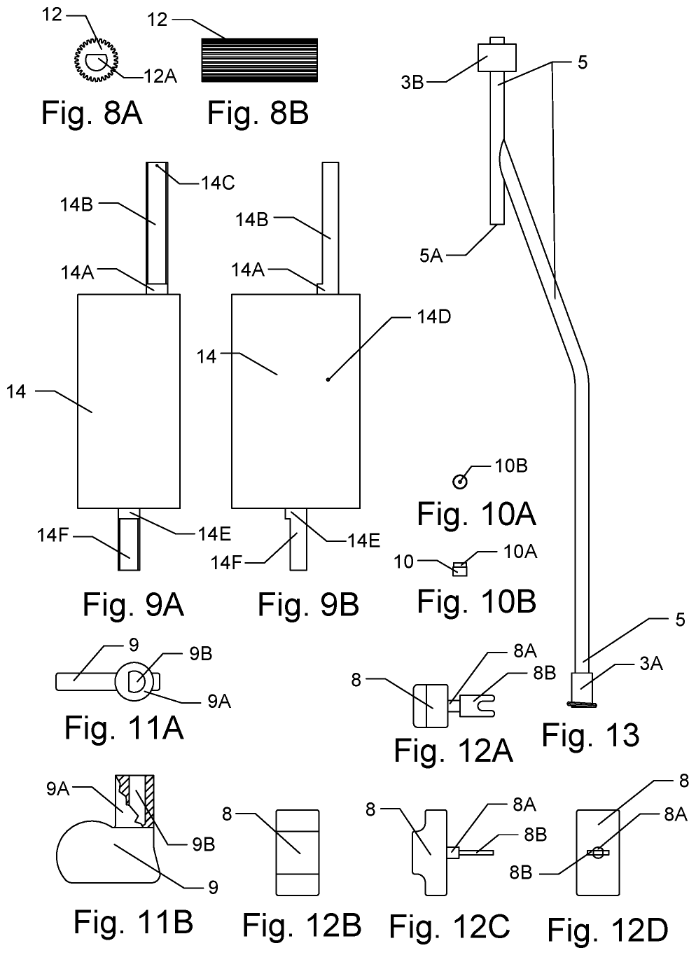

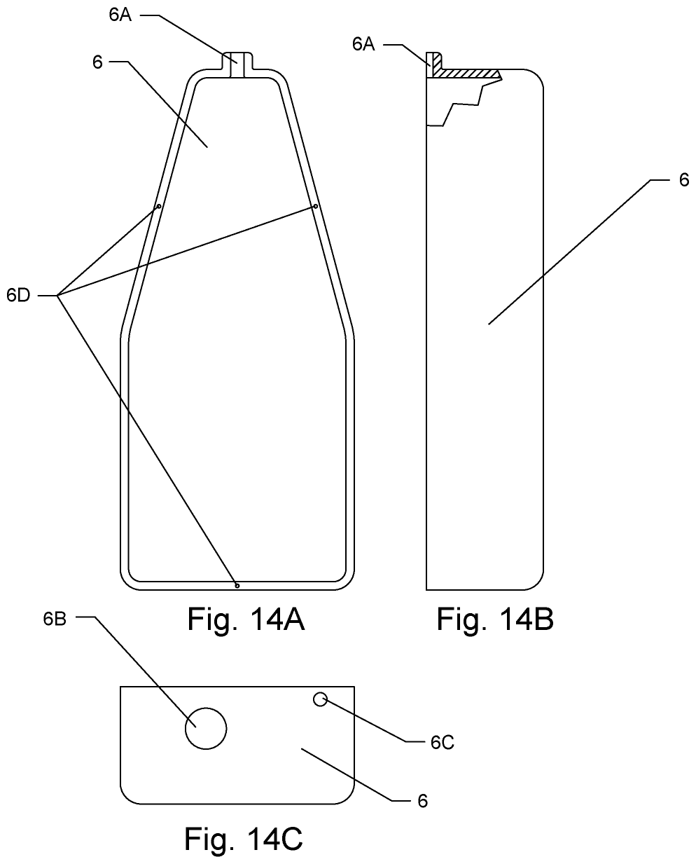

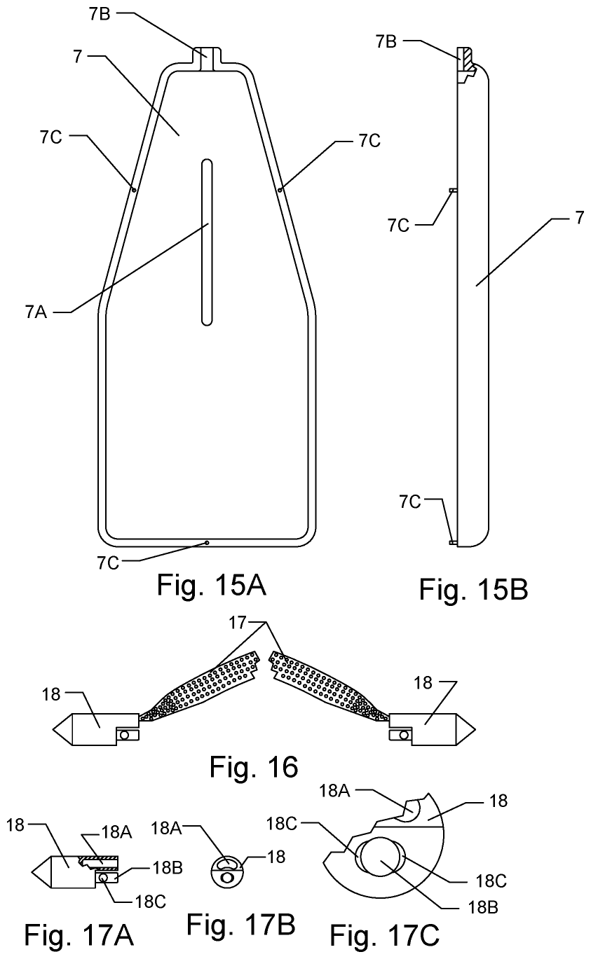

Referring to FIG. 2A, FIG. 2B, FIG. 2C, FIG. 2D, FIG. 2G, FIG. 2E, FIG. 2F, FIG. 2H, FIG. 2J, FIG. 2K, FIG. 7A, FIG. 7B, FIG. 7C, FIG. 8A, FIG. 8B, FIG. 9A, FIG. 9B, FIG. 10A, FIG. 10B, FIG. 11A, FIG. 11B, FIG. 12A, FIG. 12B, FIG. 12A, FIG. 12A, FIG. 12B, FIG. 12C, FIG. 12D, FIG. 13, FIG. 14A, FIG. 14B, FIG. 14C, FIG. 15A, and FIG. 15B in accordance with the present invention, there is a bottom-part handle 6 and there is a top-part handle 7. The bottom-part handle 6 has on its wall edges blind holes 6D while the top-part handle 7 has on its wall edges matching bosses 7C, which bosses go into the blind holes 6D of the bottom-part handle 6 for aligning and assembling the bottom-part handle 6 and top-part handle 7. The bottom-part handle 6 has on the front end a semicircular channel 6A and has on the back end a large hole 6B and a small hole 6C, while the top-part handle 7 has on the front end a matching semicircular channel 7B and has on the planar surface an elongated slot 7A.

The bottom-part handle 6 contains a boxed spring-wound motor 14, which motor has on one end, a motor drive shaft 14A with an extended motor drive D-shape shaft 14B, which shaft contains a cross hole 14C. The boxed spring-wound motor 14 on the opposite end has a motor crank shaft 14E with an extended motor crank D-shape shaft 14F and on the top has a small hole 14D. A motor drive gear 12 has a D-hole 12A and is installed via the D-hole 12A on the extended motor drive D-shape shaft 14B. A press-fit pin 15 is fitted into the cross hole 14C to retain the motor drive gear 12. The boxed spring-wound motor 14 with the installed motor drive gear 12 is placed and adhered inside to the floor of the bottom-part handle 6 while internally inserting the extended motor crank D-shape shaft 14F into the large hole 6B of the bottom-part handle 6.

A Y-branched tubular aspiration channel 5, which channel has a straight leg and an integral angled leg, integrally contains a plug luer lock 3B at the front end of the straight leg and contains at the back end 5A of the straight leg an elastomeric seal plug 10, which plug has a step 10A and a sealing hole 10B, while the step 10A is glued into the back end 5A. The straight leg of the Y-branched tubular aspiration channel 5 further contains a coupler shaft 4, which shaft at the front end has prongs 4A, which prongs have through holes 4B. The coupler shaft 4, at the front end, extends out of the plug luer lock 3B. The prongs 4A and the holes 4B together perform a coupling function by engaging. The coupler shaft 4 at the back end extends out of the sealing hole 10B of the seal plug 10 and has at the back end an extended D-shape shaft 4C, which D-shape shaft has a cross hole 4D. The extended D-shape shaft 4C contains a cutter gear 11, which gear has a D-hole 11A for the extended D-shape shaft 4C. The cutter gear 11 has a hub 11B and a concentric circular flange 11C. The coupler shaft 4 at the back end also contains an extension spring 13, which spring has a front hook 13A and a back hook 13B, while the front hook 13A is in the cross hole 4D to retain the cutter gear 11.

The straight leg of the Y-branched tubular aspiration channel 5 containing the integral plug luer lock 3B, the glued elastomeric seal plug 10, the coupler shaft 4, the cutter gear 11 and the extension spring 13 is placed on and adhered to the semicircular channel 6A of the bottom-part handle 6 while inserting the angled leg of the Y-branched tubular aspiration channel 5 into the small hole 6C of the bottom-part handle 6 and gluing the angled leg of the Y-branched tubular aspiration channel 5 to the small hole 6C of the bottom-part handle 6 and while making sure that the cutter gear 11 and motor drive gear 12 are meshed. Another receptacle luer lock 3A that functions as an aspiration tubing connection is adhered to the back end of the angled leg of the Y-branched tubular aspiration channel 5. The back hook 13B of the extension spring 13 is inserted into the small hole 14D of the boxed spring-wound motor 14. The extended motor crank D-shape shaft 14F contains a motor crank knob 9, which knob has a cylindrical feature 9A with a D-hole 9B for the extended motor crank D-shape shaft 14F. The motor crank knob 9 is fixed by placing an adhesive 16 on the extended motor crank D-shape shaft 14F while inserting the motor crank knob 9 from the cylindrical feature 9A into the large hole 6B located on the back of the bottom-part handle 6.

The top-part handle 7 contains a slider knob 8 in the elongated slot 7A, which knob has a cylindrical stem 8A with a yoke 8B. The slider knob 8 is captivated by the yoke 8B by turning the slider knob 8 to a 90° angle after inserting the slider knob 8 into the elongated slot 7A.

The bosses 7C, the semicircular channel 7B of the top-part handle 7 containing the slider knob 8 with the yoke 8B are, respectively, aligned and joined together with the blind holes 6D and the semicircular channel 6A of the bottom-part handle 6 containing all the parts mentioned above while making sure that the yoke 8B of the slider knob 8 is aligned with the hub 11B of the cutter gear 11 and is also in contact with the back of the concentric circular flange 11C. The joined top-part handle 7 and the bottom-part handle 6 are externally glued together, becoming an assembled handle 2X.

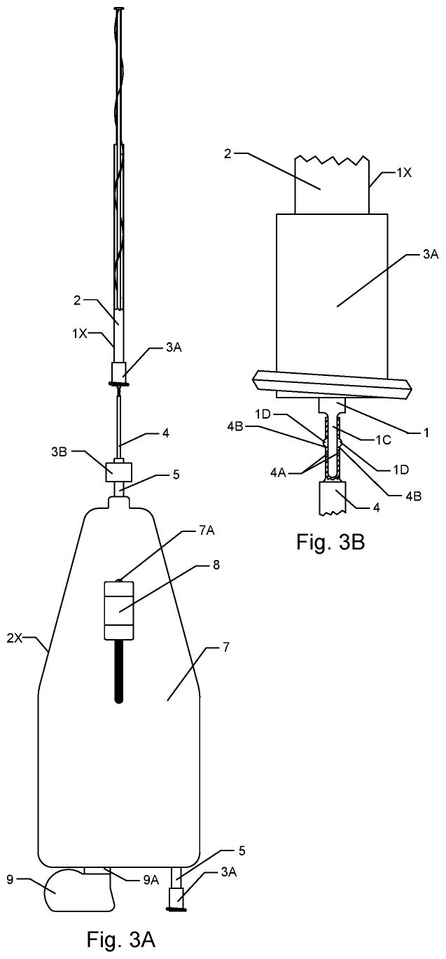

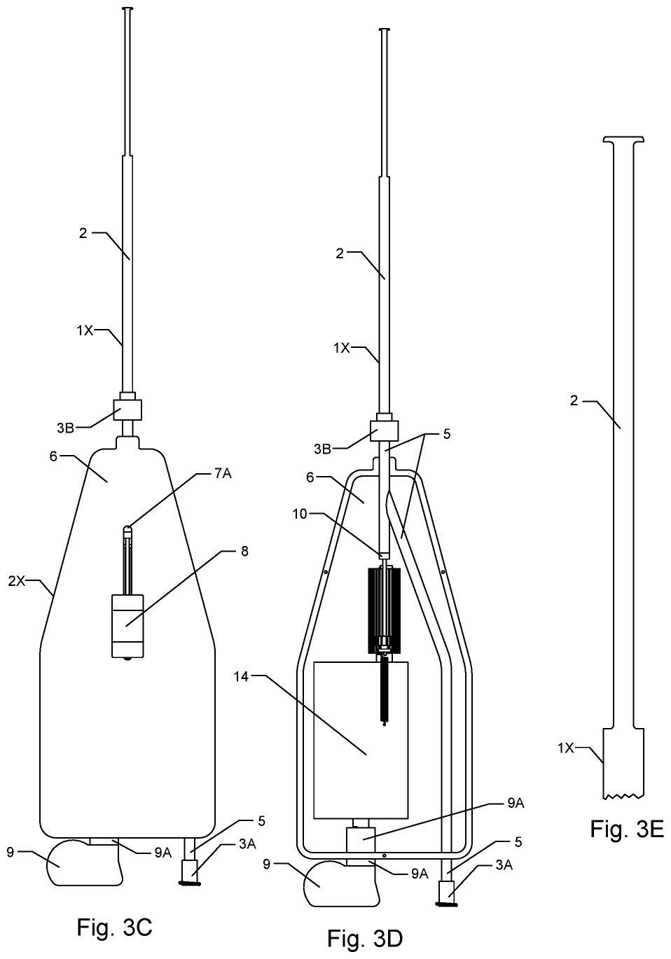

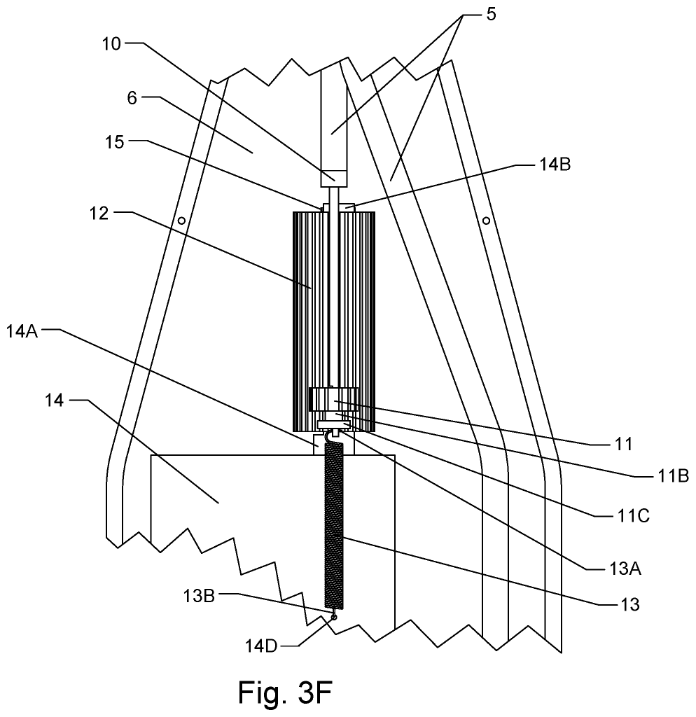

Referring to FIG. 3A, FIG. 3B, FIG. 3C, FIG. 3D, FIG. 3E, and FIG. 2F in accordance with the present invention, before starting a surgical procedure that involves resecting biological tissues and aspirating the resected tissues and any biological debris, the coupler shaft 4 is fully exposed by sliding the slider knob 8 on the assembled handle 2X all the way toward the front; and while holding the slider knob 8 at that position, the cutter shaft 1 of the helical tissue cutter 1X is coupled with the coupler shaft 4 by engaging the flattened end 1C and the rounded bosses 1D of the cutter shaft 1, respectively, with the prongs 4A and the holes 4B of the coupler shaft 4; and then the hollow cylinder 2 of the helical tissue cutter 1X is attached to the assembled handle 2X by interconnecting the plug luer lock 3B and the receptacle luer lock 3A. After removing the hold, the slider knob 8 retracts the cutter shaft 1, which shaft is coupled. This retraction allows the sharp-edged helical blade 1A to move away from the slots 2A of the hollow cylinder 2 of the helical tissue cutter 1X so as to keep the sharp-edged helical blade 1A unexposed, hence providing protection to the user and/or patient while the helical tissue cutter 1X is not in use. Now, a standard vacuumized tissue collector (not shown) containing a standard aspiration tubing (not shown) is attached to the assembled handle 2X by connecting the aspiration tubing to the receptacle luer lock 3A located at the back of the assembled handle 2X.

To resect biological tissues and aspirate the resected tissues and any biological debris, the vacuum system (not shown) of the tissue collector (not shown) is turned on and the motor crank knob 9 located at the back of the assembled handle 2X is fully turned to power the boxed spring-wound motor 14, which speedily rotates the cutter shaft 1 containing the sharp-edged helical blade 1A and the flat-edged helical fin 1B of the helical tissue cutter 1X. While it is running, the helical tissue cutter 1X is placed on a targeted surgical site (not shown) while making sure that at least one of the slots 2A of the hollow cylinder 2 is intimately in contact with the tissues of the targeted surgical site (not shown) and that the slider knob 8 on the assembled handle 2X is slid all the way toward the front and held at that position, which act exposes the rotating sharp-edged helical blade 1A through the slot 2A of the hollow cylinder 2 of the helical tissue cutter 1X in order to start resecting the tissues while spiraling out and aspirating the resected tissues and any biological debris. After resecting the tissues, the hold on the slider knob 8 is removed which causes the slider knob 8 to retract, which, in turn, moves the rotating sharp-edged helical blade 1A away from the slot 2A for safety.

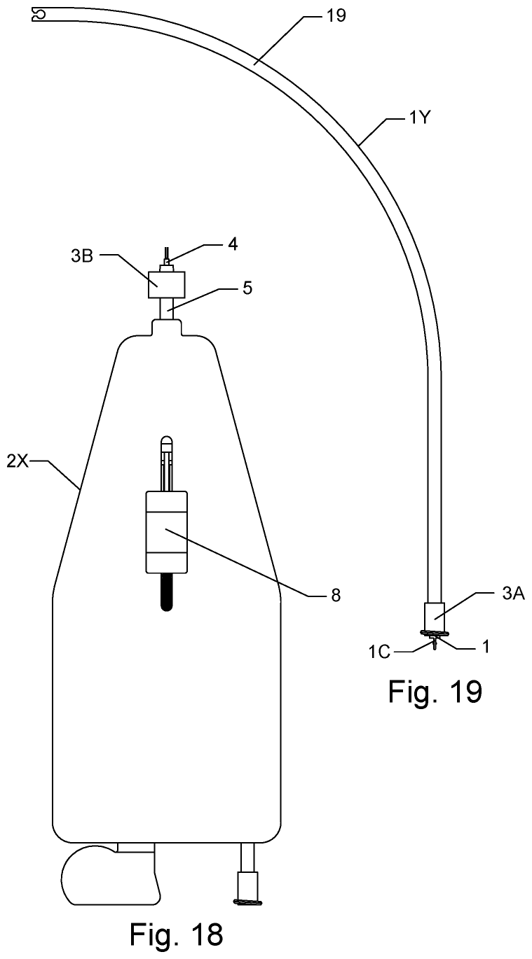

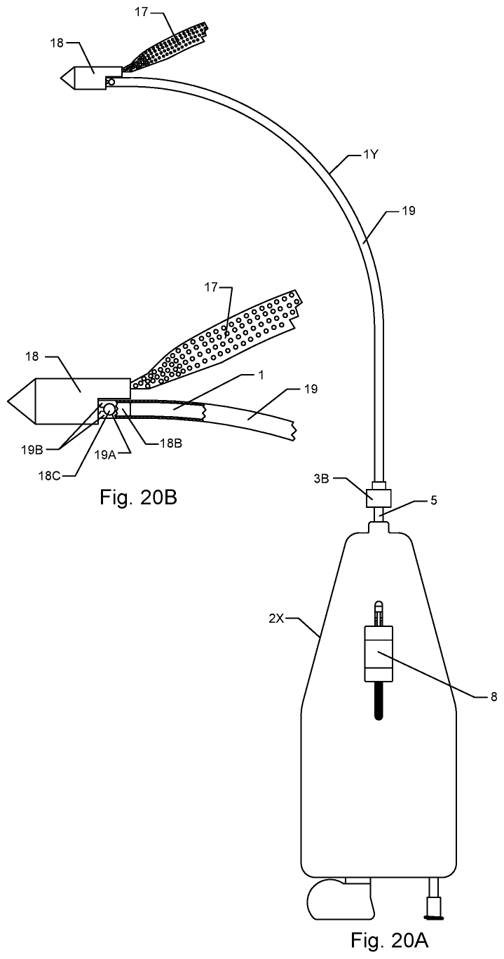

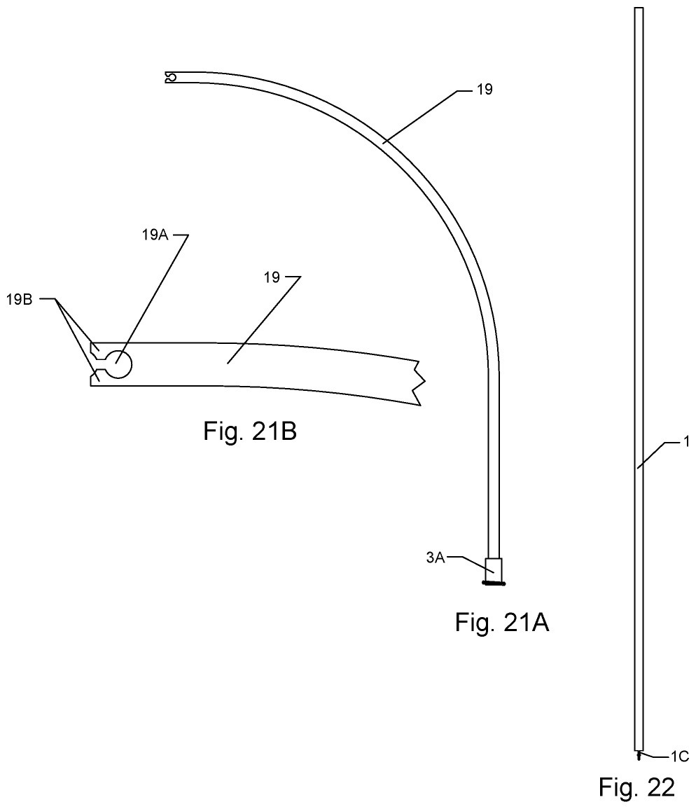

Referring to FIG. 16, FIG. 17A, FIG. 17B, FIG. 17C, FIG. 18, FIG. 19, FIG. 20A, FIG. 20B, FIG. 21A, FIG. 21B, and FIG. 22 in accordance with the present invention, for a treatment of stress urinary incontinence, a mesh sling 17 has an anchor 18 glued on the either end of the mesh sling 17 while the anchor 18 on one end has a crescent slot 18A, which slot has the glued end of the mesh sling 17. The anchor 18 on the same end also has a stud 18B, which stud has two rounded bosses 18C, one on each side of the stud 18B. A tubular curved trocar 1Y has a curved tube 19, which tube internally contains a slidable shaft that is the same as the cutter shaft 1, and hence this is now a slidable shaft 1 in this case, which shaft conforms to the curved tube 19 while being inside the curved tube 19 and which shaft has at the back the flattened end 1C and on either side of the flattened end 1C is a rounded boss 1D, which boss performs a coupling function by engaging. At the front end, the curved tube 19 has a cross holes 19A and a slit and compliant tip 19B and at the back end, the curved tube 19 contains yet another receptacle luer lock 3A. The anchor 18 with the mesh sling 17 is installed on the curved tube 19 by inserting the stud 18B with the round bosses 18C into the tip 19B of the curved tube 19 all the way until the round bosses 18C of the anchor 18 fully engage with the cross holes 19A of the curved tube 19.

As before, the coupler shaft 4 is fully exposed by sliding the slider knob 8 on the assembled handle 2X all the way toward the front; and while holding the slider knob 8 at that position, the slidable shaft 1 of the tubular curved trocar 1Y is coupled with the coupler shaft 4 by engaging the flattened end 1C and the rounded bosses 1D, respectively, with the prongs 4A and the holes 4B of the coupler shaft 4; and then the tubular curved trocar 1Y is attached to the assembled handle 2X by interconnecting the plug luer lock 3B and the receptacle luer lock 3A.

Using the retropubic approach, after creating periurethral tunnels with the helical tissue cutter 1X, the mesh sling 17 with the anchor 18 installed on the tubular curved trocar 1Y is deployed intravaginally and then the installed anchor 18 is released from the tubular curved trocar 1Y by sliding the slider knob 8 on the assembled handle 2X all the way toward the front, which dislodges the anchor 18 with the mesh sling 17.

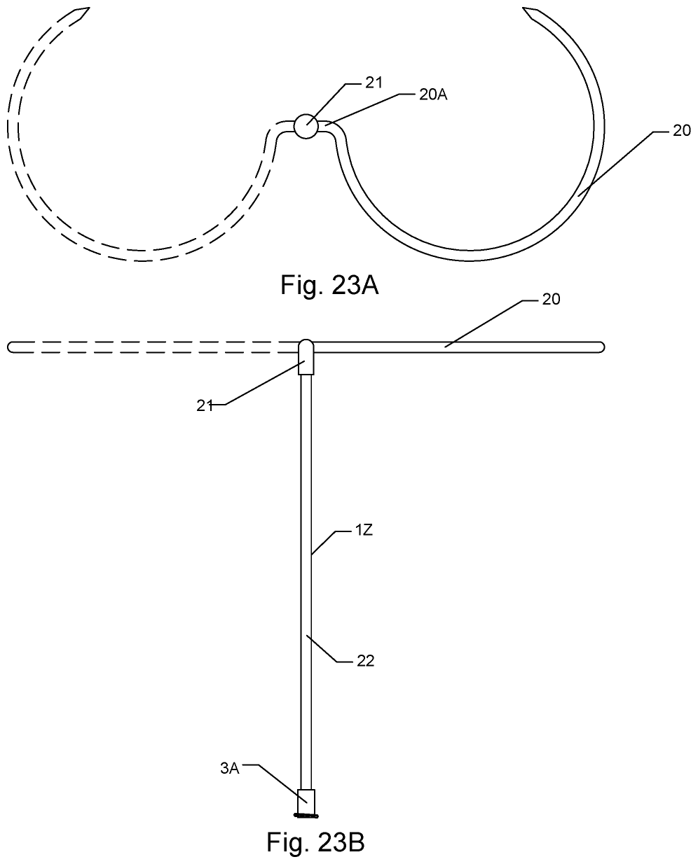

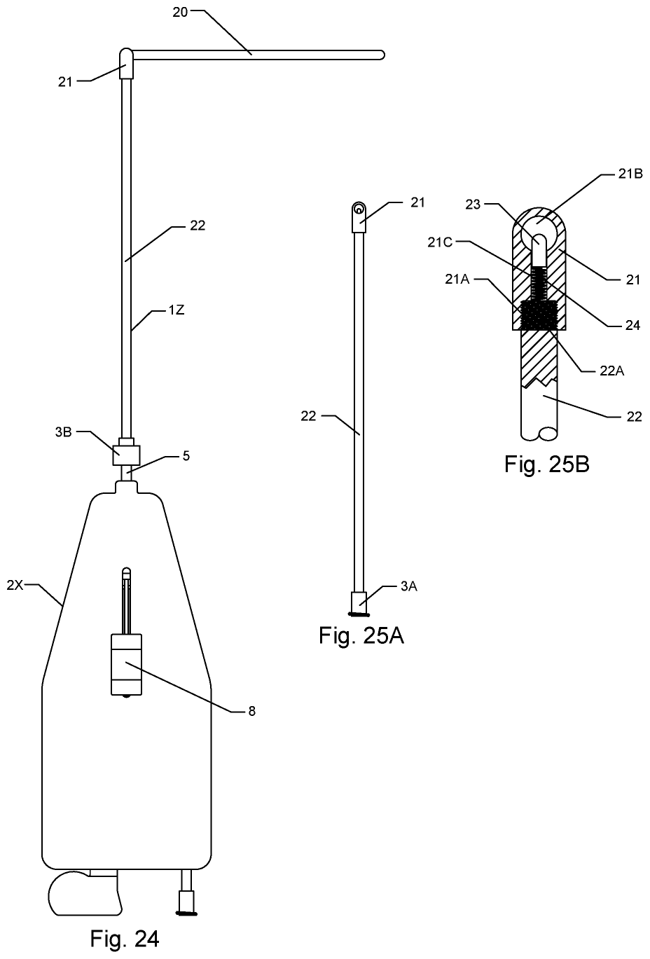

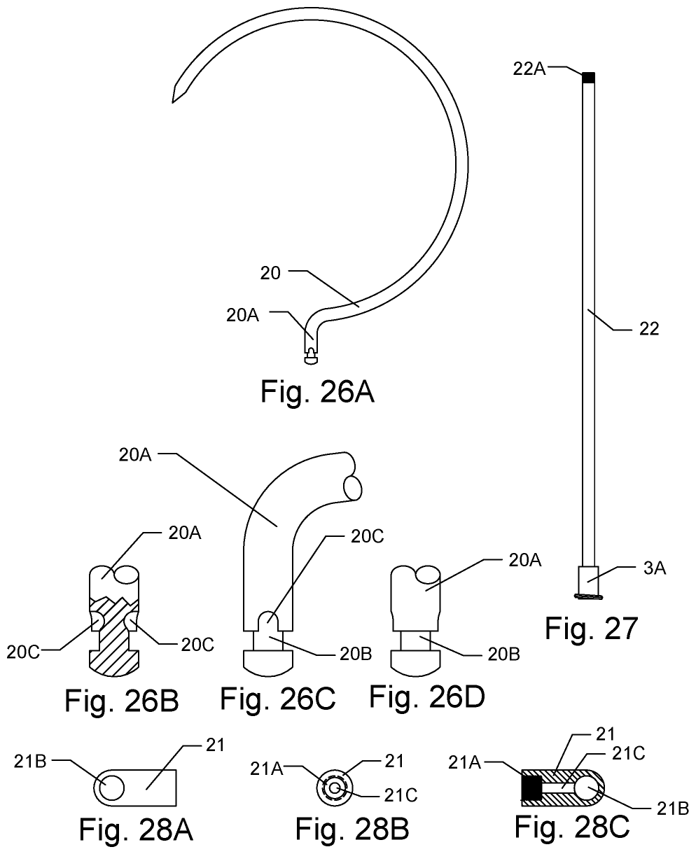

Referring to FIG. 23A, FIG. 23B, FIG. 24, FIG. 25A, FIG. 25B, FIG. 26A, FIG. 26B, FIG. 26C, FIG. 26D, FIG. 27, FIG. 28A, FIG. 28B, and FIG. 28C in accordance with the present invention, a turnable trocar 1Z contains a curved trocar wire 20, which wire forms on one end a planar stem 20A, which stem has a groove 20B. On one edge of the groove 20B are two notches 20C, which notches are located 180° apart. A receptacle stud 21 has at one end a cross hole 21B, which hole contains the stem 20A of the curved trocar wire 20. The receptacle stud 21 has on the other end a blind axial hole 21C and a larger concentric threaded hole 21A. The blind axial hole 21C, which intersects the cross hole 21B, houses a plunger 23 and a compression spring 24, both of which are parts of a turn-lock mechanism for the turnable trocar 1Z. The plunger 23 captivates the curved trocar wire 20 via the engagement of the plunger 23 with the groove 20B and the notches 20C on the stem 20A. The larger concentric threaded hole 21A contains a trocar shaft 22 with threads on one end 22A, which end is inserted into the threaded hole 21A. The trocar shaft 22 on the other end has still another receptacle luer lock 3A.

The curved trocar wire 20 can freely and fully turn when the plunger 23 is in the groove 20B on the stem 20A while the curved trocar wire 20 is lifted, but to lock its turn, the curved trocar wire 20 is pushed down to feelingly find and engage the plunger 23 with one of the two notches 20C, resulting a lockingly turnable trocar 1Z. As before, the turnable trocar 1Z is attached to the assembled handle 2X by interconnecting the plug luer lock 3B and the receptacle luer lock 3A.

With a transobturator approach and after creating any needed periurethral tunnels with the helical tissue cutter 1X, the turnable trocar 1Z is used for a right or left side of the groin as a single tool to treat stress urinary incontinence.

The invention claimed is:

1. A helical tissue cutter, to be used for resecting biological tissues and for spiraling out the resected tissues and any biological debris, having an enclosed, exposable, retractable, protective cutter with a helical blade that is continued further as a helical fin, said cutter comprising:
an elongated shaft longitudinally having said helical blade continued further a as said helical fin on the cylindrical surface around the neutral axis of said shaft, said blade having a sharp edge, said fin having a flat edge, said shaft having a flattened end, and said end having a rounded boss on both flat sides of said end to perform a coupling function by engaging;
an elongated hollow cylinder for enclosing said cutter, said cylinder longitudinally having at least two opposing elongated through slots for exposing said blade on the sides of said cylinder when in use, said cylinder having one end open, said end having bent and rounded edge, and said cylinder containing at the other end a receptacle luer lock to perform an attachment function.

2. A tubular curved trocar having a capability and means to intravaginally carry, deploy and release a mesh sling using a retropubic approach to treat stress urinary incontinence, said trocar comprising:
a curved tube having on one end a cross hole and a slit, and said tube having at the other end said receptacle luer lock;
a shaft being conformable to and residing within said curved tube, said shaft having a flattened end, and said end having a rounded boss on both flat sides of said end to perform a coupling function by engaging;
a rod functioning as an anchor for a mesh sling, said anchor having at one end a pointed shape and at the other end a deep crescent slot and a stud, said anchor having one end of the mesh sling bonded into said slot, said stud having orthogonally at least one rounded boss, and said boss engaging into said cross hole of said tube.

3. A turnable trocar that can be used on the right or left side of the groin to deploy a mesh sling with a transobturator approach to treat women's stress urinary incontinence, said turnable trocar comprising:
a curved trocar wire having a planar stem, said stem having a groove, said groove having at one edge two notches, and said notches being 180° apart;
a receptacle stud having a cross hole at one end, said receptacle stud having axially on the other end a blind hole and a larger concentric threaded hole, and said blind hole intersecting said cross hole;
a plunger residing in said blind hole, and said plunger engaging with said groove and said notches;
a compression spring residing in said blind hole, and said spring mating with said plunger;
a trocar shaft having threads on one end and said receptacle luer lock on the other end.

4. A handle having a motorized capability and means to couple, attach, handle and operate, slide and retract said cutter while having an aspiration connection for a vacuumized tissue collector, or to couple, attach, handle and operate said tubular curved trocar or said turnable trocar, said handle comprising:
a top part and a bottom part, said top part having a shallow-trough shape with alignment bosses and a semicircular channel on the wall edges and an elongated slot on the planar surface, said bottom part having a matching deep-trough shape with matching alignment blind holes and a matching semicircular channel on the wall edges, and said bottom part on the back wall having a large hole and a small hole;
a slider knob orthogonally having a cylindrical stem, said stem having on the end a flat yoke, said knob being contained in said slot of said top part, and said yoke captivating said knob in said top part when said knob turned to a 90° angle;
an elongated motor drive gear having a D-hole;
a boxed spring-wound motor having a small hole on the top and having a D-shape motor drive shaft on the front and a D-shape crank shaft on the back, said drive shaft having a cross hole, said drive shaft having said drive gear and a press-fit pin in said cross hole, said pin retaining said drive gear, said crank shaft passing through said large hole of said bottom part, and said boxed motor being bonded internally to the floor of said bottom part;
an elastomeric plug having a step, and said plug having a through sealing hole;
a Y-branched tube having a straight tube and a joined angled tube, said branched tube functioning as an aspiration channel, said straight tube being bonded to said semicircular channel of said bottom part and said angled tube passing through and bonded to said small hole of said bottom part, said straight tube on one end having said plug bonded and on the other end having a plug luer lock for attaching said cutter or said tubular curved trocar or said turnable trocar via said receptacle luer lock, said angled tube at the end having said receptacle luer lock, and said receptacle luer lock connecting to a standard or conventional aspiration tubing attached to a conventional or standard vacuumized tissue collector;
an elongated extension spring having a hook on either end;
a cutter drive gear having a D-hole, said cutter gear having a hub, and said hub having a concentric circular flange;
a coupler shaft at the front end having prongs, said prongs having through holes for performing a coupling function by engaging with said rounded bosses of said cutter or said trocar, said coupler at the back end having a D-shape shaft, said D-shape shaft having a cross hole, said coupler going through said straight tube and extending out of said plug luer lock at said front end and also extending out of said sealing hole of said plug at said back end, said coupler having said cutter gear on said D-shape shaft, said D-shape shaft having said hook of said extension spring in said cross hole, said hook retaining said cutter gear, said boxed motor having the other said hook of said extension spring in said hole of said boxed motor;
a crank knob having a cylindrical stem, said stem having a blind D-hole, and said crank knob being on and bonded to said crank shaft of said boxed motor while said stem going through said large hole of said bottom part.
